(19) United States
(12) Patent Application Publication
(10) Pub. No.: US 2025/0061530 A1
(43) Pub. Date: Feb. 20, 2025

(54) COMPUTER IMPLEMENTED SYSTEM AND METHOD FOR DETERMINING EDUCATIONAL PROFICIENCY AND LEARNING DISABILITIES

(71) Applicant: Robyn KING, Pearce (AU)

(72) Inventor: Robyn KING, Pearce (AU)

(21) Appl. No.: 18/720,121

(22) PCT Filed: Dec. 7, 2022
(86) PCT No.: PCT/AU2022/051462

§ 371 Date: Jun. 14, 2024

(30) Foreign Application Priority Data
Dec. 15, 2021 (AU) 2021904079

(51) Int. Cl. G06Q 50/20; G09B 5/12; G16H 50/20

(52) U.S. Cl. CPC G06Q 50/205; G09B 5/12; G16H 50/20

(57) ABSTRACT

The present invention relates to a computer-implemented system for facilitating the collection and analysis of academic assessment data, the system including one or more processors in operable communication with one or more devices, each device operable by a student and configured to connect with a data communications network such that any information requested of, or received by, the student, is retrieved from, and/or stored in, a data repository connected to the data communications network, the one or more processors operable to request information from the student in accordance with an assessment, receive data entered by the student using their device in response to the request for information, retrieve data stored in the data repository associated with the student, perform an analysis upon the received data from the student and the retrieved data associated with the student to determine the education proficiency of the student according to the assessment and further assessing the presence of one or more indicators regarding one or more learning disorders associated with the student, and generate a report regarding the educational proficiency and/or presence of the one or more indicators for subsequent storage and/or transmission.

### CROSS-REFERENCE TO RELATED APPLICATIONS

This application is a national stage entry of International Application No. PCT/AU2022/051462 filed on Dec. 7, 2022, which claims the benefit of priority to Australian Application No. 2021904079, filed Dec. 15, 2021, the entire disclosures of which are hereby incorporated herein by reference.

### FIELD OF THE INVENTION

The present invention relates to a system and method for collecting data regarding on-line learning assessments and analysing same. The system and method is particularly useful for maintaining a record of an individual's progress regarding their learning journey and may also be useful when seeking to identify learning disabilities that are typically difficult to identify and assess in situations that require consistent and regular monitoring of an individual's progress over an extended period of time, for example, over a number of years.

### BACKGROUND OF THE INVENTION

Traditional methods of monitoring a student's academic progress in a particular field of study involve requiring the student to undertake one or more assessments with a view to understanding the student's knowledge based solely on the student's responses to a particular assessment in isolation of other factors, including their performance in other areas of study and in different educational institutions.

Due to the isolated nature of traditional forms of assessment, an assessor is only able to gain a limited insight into a student's academic progress in a particular field of study. Moreover, when a student underperforms in an assessment, the assessor often concludes, incorrectly, that the student has failed to apply themselves. In other words, this factor is considered the sole basis for their underperformance in an assessment, and fails to give consideration to other possible reasons for the student's poor, or suboptimal, performance (e.g. one or more underlying learning disorders).

Furthermore, as more learning activities and assessments are moving from a classroom environment toward an online environment, teachers do not have the benefit of face-to-face interaction with their students and thereby often miss subtle queues and indicators regarding learning difficulties/disorders or particular problems with specific types of subject matter that may impede or retard a student's academic progress.

Existing systems and methods of learning and assessment, particularly when conducted in an online environment, do not facilitate assessors identifying one or more indicators of underperformance including disabilities or learning disorders of students. In particular, existing systems and methods of assessing a student's academic progress do not take into account, or are unable to take into account, a student's individual learning difficulties, learning styles or any learning disabilities the student may have (e.g. dyslexia, disgraphia, dyscalcula, auditory processing disorders, language processing disorders, nonverbal learning disabilities and/or visual perceptual/visual motor deficit).

Accordingly, there exists a need for a system and method that addresses the abovementioned problems associated with existing learning and assessment systems and methods, or at least provides an alternative to same. More particularly, there exists a need for a system and method that enables the automated collection and analysis of data to assist the identification of academic underperformance including disabilities and learning disorders in students in a consistent and objective manner, and thereby assist rectification such that students realise their full academic potential.

The reference to any prior art in this specification is not, and should not be taken as, an acknowledgement or any suggestion, that the prior art forms part of the common general knowledge.

### SUMMARY OF THE INVENTION

In one aspect, the present invention provides a computer-implemented system for facilitating the collection and analysis of academic assessment data, the system including one or more processors in operable communication with one or more devices, each device operable by a student and configured to connect with a data communications network such that any information requested of, received by, or relating to the student, is retrieved from, and/or stored in, a data repository connected to the data communications network, the one or more processors operable to request information from the student in accordance with an assessment, receive data entered by the student using their device in response to the request for information, the data entered by the student including any one or more of, a selected option from a number of options presented to the student, a hand-written text or numeric answer, a text or numeric answer entered using a keyboard, and a voice capture, receive additional data from one or more assessors associated with the student as the student progresses through their educational journey from the commencement of primary school through to their final year of high school, receive current and/or historical information relating to educational instructions attended by the student, retrieve data stored in the data repository associated with the student, perform an analysis upon the received data from the student, the retrieved data associated with the student, the additional data received from the one or more assessors, and the current and/or historical information relating to educational institutions attended by the student, to determine the education proficiency of the student according to the assessment and further identifying the presence of one or more indicators regarding one or more learning disorders associated with the student, wherein the analysis is performed by a voice recognition algorithm for assessment of word and sound pronunciation of voice recorded data, and any one or more of a reading analysis algorithm for assessment of a reading proficiency, a spelling analysis algorithm for assessment of spelling proficiency, or a mathematical analysis algorithm for assessment of mathematical proficiency, wherein each of the algorithms operate according to a benchmark associated with a particular question or activity, and generate a report regarding the determined educational proficiency and/or presence of the one or more identified indicators for subsequent storage and/or transmission, and further automatically adjust any future assessments to suit the determined educational proficiency and/or the one or more identified indicators regarding one or more learning disorders associated with the student.

Embodiments of the present invention may find particular application with respect to on-line learning arrangements wherein students receive and complete educational assessments using a computing device (e.g. computer tablet) that collects data for each student with the data subsequently analysed using a reading, spelling and/or mathematical analysis algorithm to determine the educational proficiency of the student and the identification of any possible learning difficulties or disabilities.

In a particular embodiment, the system is used by students from a very young age (e.g., kindergarten) through to their final year of high school and whilst collecting data for each student as they progress through their educational journey, any data and/or analysis is collected and stored in the data repository for sharing with future teachers. It will be appreciated that the sharing of such data may be particularly useful if a student transfers from one school to another or transitions from primary school to high school or high school to tertiary education.

In an embodiment, the analysis may include the implementation of one or more algorithms operable to determine the presence of one or more indicators regarding one or more learning difficulties or disorders associated with the student.

In an embodiment, the one or more indicators may relate to one or more learning difficulties or disorders with respect to reading, spelling and mathematics.

In an embodiment, the data collected by the system may include recorded voice data, and the analysis includes voice recognition algorithms such that word and sound pronunciation may be assessed.

In an embodiment, the data stored in the data repository may be made available for review by the student and/or assessors.

In an embodiment, the system may perform analysis to determine the presence of indicators regarding learning difficulties or disorders including, but not limited to, dyslexia, disgraphia, dyscalcula, auditory processing disorders, language processing disorders, nonverbal learning disabilities and visual perceptual/visual motor deficit.

In an embodiment, the data repository stores further information regarding the expected education proficiency for individual students of different ages and the one or more processors apply the educational proficiency expected for individual students and compare same with an actual education proficiency of each student determined from the assessments.

In an embodiment, the one or more processors are further operable to adjust assessments to suit the educational proficiency of individual students according to individual requirements, disabilities or learning disorders of the students as identified by the system.

In an embodiment, the one or more processors are operable to provide the assessors and/or parents/guardians with one or more notifications identifying notable events with respect to the assessment of a student.

In an embodiment, the one or more processors are operable to conduct various trend analyses with respect to student assessment in order to determine individual student progress and identify trends that will likely result in difficulty for a student to achieve an expected educational proficiency in the future. In this regard, it is often the case that whilst assessors are able to identify significant events that indicate a potential learning disorder, often learning disabilities/disorders in an individual are not accompanied by any significant signs or queues, and are therefore missed. This is particularly the case if the progress of a student's educational proficiency is not followed/monitored over a sufficient period of time in an objective and consistent manner.

In this regard, it will be appreciated that a number of factors may affect an assessor's ability to detect one or more indicators of a disability or learning disorder of a student. Such factors include, but are not limited to, the assessor's level of training, skill and expertise, or an assessor's ability to perceive an indicator of a disability or learning disorder. Accordingly, depending on the assessor, different (and sometimes incorrect) conclusions may be formed regarding a student's academic proficiency and thereby suboptimal remedies and courses of action may be suggested in an attempt to facilitate a student's academic progress.

The system and method of the present invention thereby seeks to provide a consistent and objective method of assessing a student's educational/academic proficiency. In particular, the system and method of the present invention may be used to assess a student's educational proficiency/academic performance over sufficient periods of time to identify queues and/or signs (indicators of any difficulties, disabilities or learning disorders associated with the student) that may otherwise be imperceptible to an assessor when teaching/assessing a student in an online environment and, in particular, when teaching/assessing the student in the absence of any knowledge regarding the student's prior academic history and performance. It will be appreciated that the ability of the present system and method to identify trends in a student's progress that may otherwise be imperceptible to an assessor relying upon their subjective skills of identifying indicators of disabilities and learning disorders, advantageously allows for early intervention and better prospects for successful learning outcomes and student academic progress.

In one particular embodiment, the analysis performed to determine the educational proficiency of the student takes into account indigenous ways of learning such that learning difficulties may be detected where the cause of difficulty relates to a difference between the students preferred learning style as compared with style of the subject matter delivery. Of course, this embodiment may be particularly useful for indigenous students and improving indigenous literacy and numeracy statistics.

It will be appreciated that in various embodiments, the system is operable to enable students and/or assessors to access the system from anywhere, and at any time, such that learning and student assessment is not restricted to any geographical location or time zone.

In another aspect, the present invention provides a computer-implemented method for facilitating the collection and analysis of academic assessment data, the method including requesting, by one or more processors associated with a computer-implemented system, information from a student in accordance with an assessment, receiving, by the one or more processors, data entered by the student in response to the request for information using a device operable to connect with a data communications network and storing the data in a data repository connected to the data communications network, the data entered by the student including any one or more of, a selected option from a number of options presented to the student, a hand-written text or numeric answer, a text or numeric answer entered using a keyboard, and a voice capture, receive additional data from one or more assessors associated with the student as the student progresses through their educational journey from the commencement of primary school through to their final year of high school, receive current and/or historical information relating to educational instructions attended by the student, retrieving, by the one or more processors, the data associated with the student stored in the data repository, performing, by the one or more processors, an analysis upon the data associated with the student, the additional data received from the one or more assessors, and the current and/or historical information relating to educational institutions attended by the student, to determine the educational proficiency of the student according to the assessment and further identifying the presence of one or more indicators regarding one or more learning disorders associated with the student, wherein the analysis is performed by a voice recognition algorithm for assessment of word and sound pronunciation of voice recorded data, and any one or more of a reading analysis algorithm for assessment of a reading proficiency, a spelling analysis algorithm for assessment of spelling proficiency, or a mathematical analysis algorithm for assessment of mathematical proficiency, wherein each of the algorithms operate according to a benchmark associated with a particular question or activity, and generating, by the one or more processors, a report regarding the determined educational proficiency and/or presence of the one or more identified indicators for subsequent storage and/or transmission, and further automatically adjust any future assessments to suit the determined educational proficiency and/or the one or more identified indicators regarding one or more learning disorders associated with the student.

In a further aspect, the present invention provides a computer-readable medium that when executed on a computer, causes one or more processors of the computer to perform the steps of request information from a student in accordance with an assessment, receive data entered by the student in response to the request for information using a device operable to connect with a data communications network and storing the data in a data repository connected to the data communications network, the data entered by the student including any one or more of, a selected option from a number of options presented to the student, a hand-written text or numeric answer, a text or numeric answer entered using a keyboard, and a voice capture, receive additional data from one or more assessors associated with the student as the student progresses through their educational journey from the commencement of primary school through to their final year of high school, receive current and/or historical information relating to educational instructions attended by the student, retrieve the data associated with the student stored in the data repository, perform an analysis upon the data associated with the student, the additional data received from the one or more assessors, and the current and/or historical information relating to educational institutions attended by the student, to determine the educational proficiency of the student according to the assessment and further identify the presence of one or more indicators regarding one or more learning disorders associated with the student, wherein the analysis is performed by a voice recognition algorithm for assessment of word and sound pronunciation of voice recorded data, and any one or more of a reading analysis algorithm for assessment of a reading proficiency, a spelling analysis algorithm for assessment of spelling proficiency, or a mathematical analysis algorithm for assessment of mathematical proficiency, wherein each of the algorithms operate according to a benchmark associated with a particular question or activity, and generate a report regarding the determined educational proficiency and/or presence of the one or more identified indicators for subsequent storage and/or transmission, and further automatically adjust any future assessments to suit the determined educational proficiency and/or the one or more identified indicators regarding one or more learning disorders associated with the student.

### BRIEF DESCRIPTION OF THE DRAWINGS

Embodiments of the invention will now be described in further detail with reference to the accompanying Figures in which:

FIG. 1 provides an overview of a system according to an embodiment of the invention showing, in particular, the interaction between various system components;

FIG. 2 illustrates an exemplary diagram associated with the server component within the system illustrated in FIG. 1;

FIG. 3 illustrates an exemplary flow diagram of the server component of FIG. 2 communicating with a software application that is accessible by a teacher/administrator for the purpose of creating and maintaining tests and assessments;

FIG. 4 illustrates an exemplary flow diagram of a process that enables the teacher/administrator to utilize their software application to manage and maintain a class profile and the scheduling of assessments;

FIG. 5 illustrates an exemplary flow diagram of a process that enables a student to download and install the software application, and subsequently access, or register to use, the software application, for interaction with the system illustrated in FIG. 1;

FIG. 6 illustrates an exemplary flow diagram of a process that enables the student to undertake a test or assessment and for the test or assessment response to be assessed; and

FIG. 7 illustrates an exemplary flow diagram of a process that enables the teacher/administrator to access the test or assessment results, including a detailed report on each student's progress.

### DETAILED DESCRIPTION OF EMBODIMENT(S) OF THE INVENTION

For simplicity and illustrative purposes, the present disclosure is described by referring to embodiment(s) thereof. In the following description, numerous specific details are set forth in order to provide a thorough understanding of the disclosure. It will be readily apparent, however, that the current disclosure may be practiced without limitation to the specific details. In other instances, some methods and structures have not been described in detail to avoid obscuring disclosure.

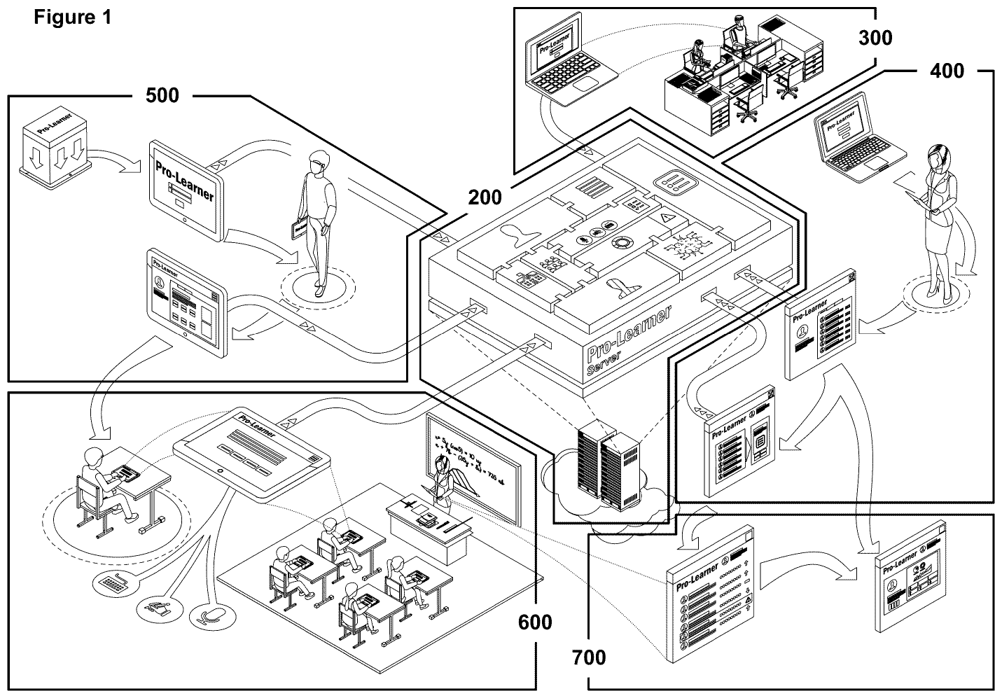

According to an embodiment, the present invention relates to a system and method for facilitating the correction and analysis of academic assessment data as depicted in FIG. 1. The system and method provide a platform that hosts a computer-executable software application (40), wherein the application (40) is accessible by a plurality of users, including student users (30) as well as teacher/administrative users (60). The platform enables the generation of a report (320) regarding the educational proficiency of a student (30) and/or the presence of one or more indicators regarding learning disabilities/disorders associated with the particular student (30), for storage and/or transmission to an assessor (60) (e.g. the teacher/administrator or a parent/guardian).

The central server (20) maintains one or more processors and/or databases for performing functions, including requesting information from a student (30) in accordance with an assessment (310), and receiving data (312/314/316) input by the student (30) in response to the request for information. The student (30) uses their data communication device (50) to input the data, which is operable to connect with the server (20) via a data communications network, the server (20) operable to store the data in a data repository (160). The server (20) may then retrieve the data associated with the student (30) and perform an analysis (e.g. using artificial intelligence analysis engine (170)) upon the data associated with the student (30) to determine the educational proficiency of the student (30) according to the assessment. The server (20) may further determine the presence of one or more indicators regarding one or more learning disorders associated with the student (30).

The person skilled in the relevant field of technology will appreciate that the platform provides a solution that provides teachers/administrators (60), and others who have an interest in the education of students (30) (e.g. parents and/or caregivers), with the ability to regularly assess the learning of the students (30). Through the use of the artificial intelligence engine (170), the users (60) may identify students who are progressing well through a syllabus, for example, as compared with those who are struggling with the content (taking into account additional factors including learning disorders associated with the student (30), etc). In this way, the application (70) may be utilized to identify learning difficulties in the student (30) so that early intervention and assistance can be provided to prevent students (30) falling behind.

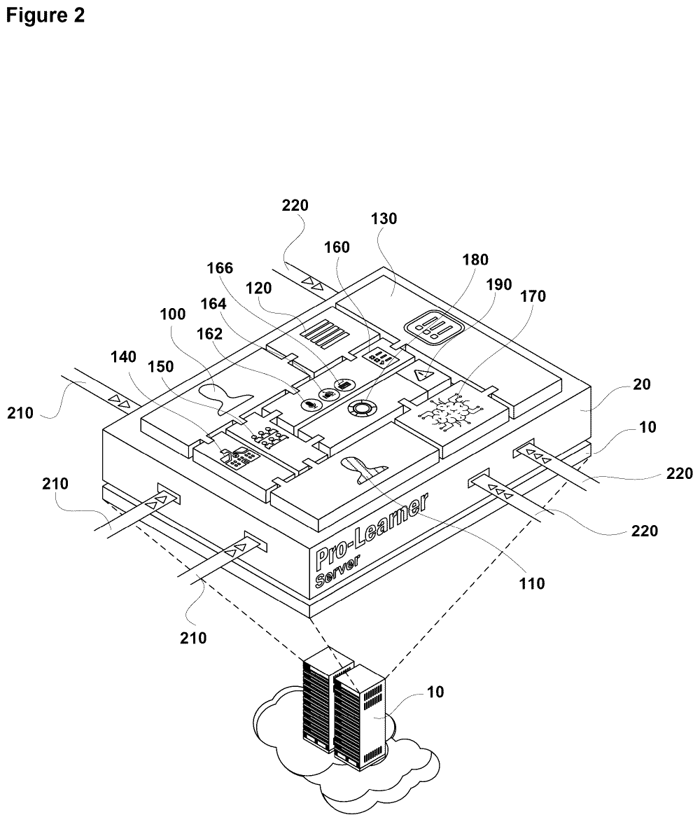

FIG. 1 is divided into segments which are further expanded in subsequent FIGS. 2 to 7. In particular, segment 200 of FIG. 1 shows the server component (20) with which a software application (40) operating on data communication devices (50) of students (30) are configured to communicate. The server component (20) is also operable to communicate with data communication devices (50) associated with the teachers/administrators (60), thereby providing the teachers/administrators (60) with access to their specific version of the software application, i.e. software application (70). It will be apparent to the person skilled in the relevant field of technology that the software applications (40/70) may be mobile applications or web applications, and similarly, the data communication devices (50) utilized by the students (30) and the teachers/administrators (60) may be mobile devices or computer devices. Examples of mobile devices include mobile phones or tablets and examples of computer devices include laptops or personal computers.

The server component (20) is additionally detailed in FIG. 2 and the skilled person will appreciate that the steps described herein, as executable by the devices (50), may be facilitated by the software applications (40/70) operating on each device (50).

As an alternative, or in addition to, steps described herein as performed by the server (20) the steps described may be performed by one or more processors associated with the user devices (50) (i.e. in a distributed architecture). Different arrangements are possible in this regard, but according to a particular implementation of the present invention, the server (20) is programmed to provide all of the functions described herein where they cannot be provided locally on the user devices (50).

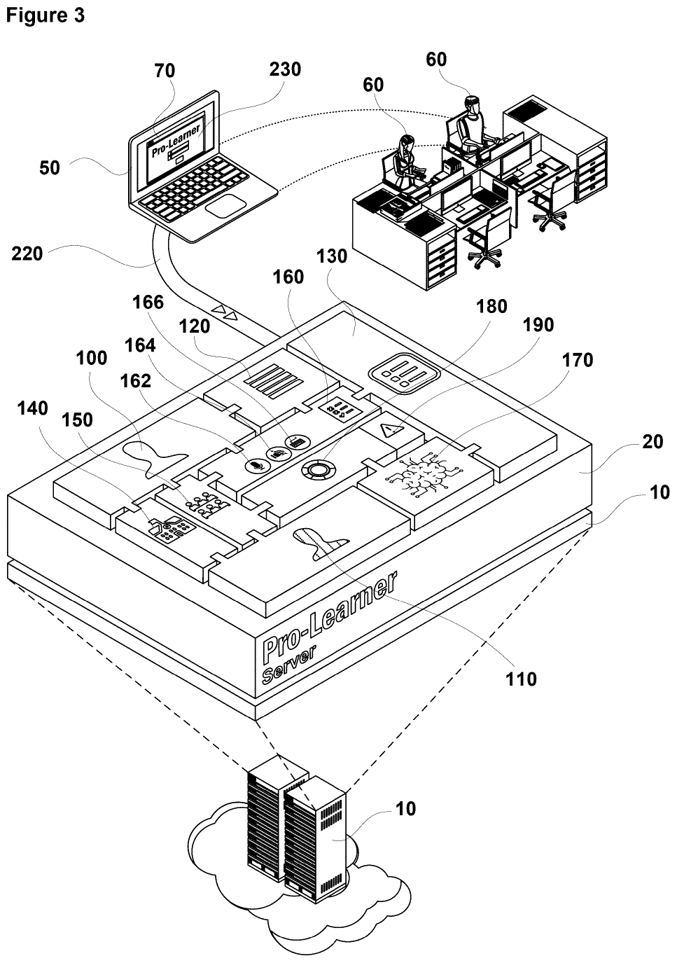

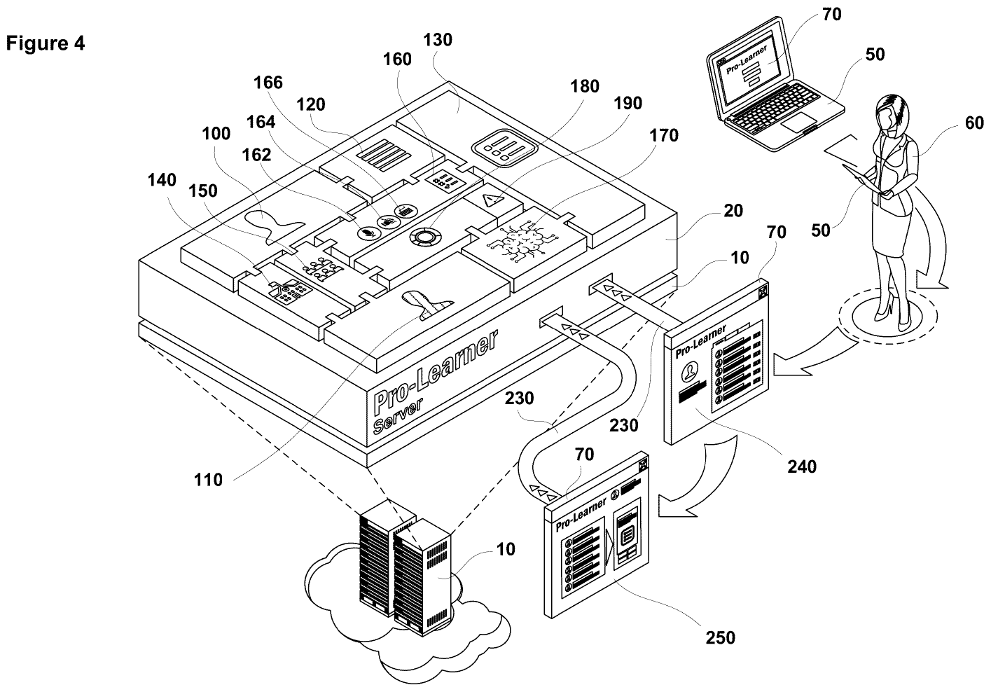

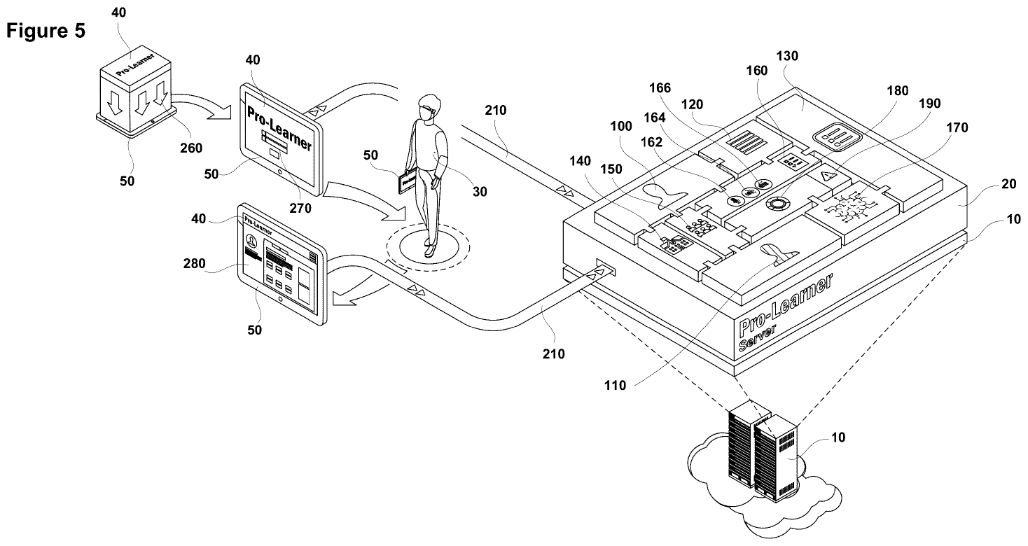

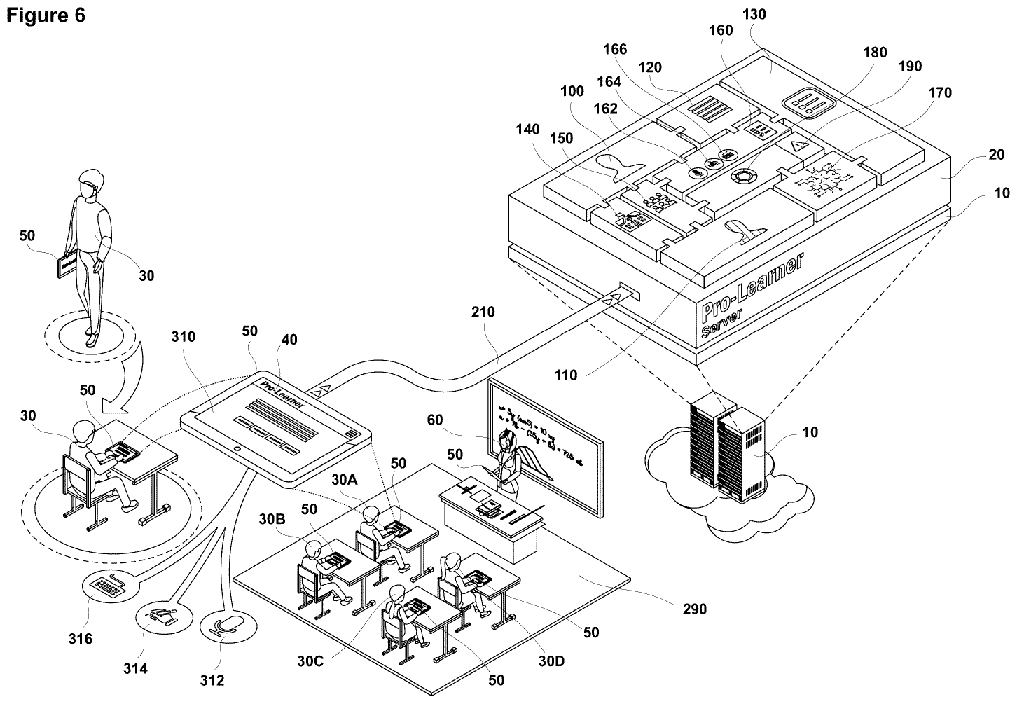

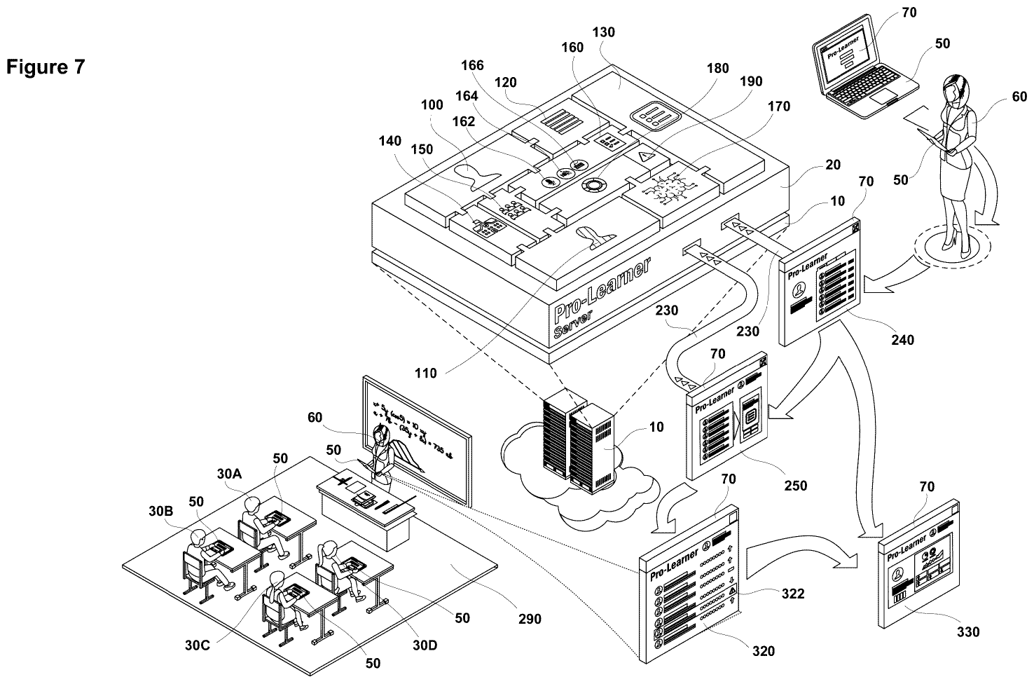

Segment 300 of FIG. 1 shows how the server (20) may be configured for communication (220) with the device (50) associated with a teacher/administrator (60) for the purpose of creating and maintaining an assessment for students (30), as further detailed in FIG. 3. Segment 400 of FIG. 1 shows how the teacher/administrator (60) may utilize the software application (70) to manage and maintain a class profile (240) and schedule an assessment (250) of some or all of the students (30) in their class, as further detailed in FIG. 4. Segment 500 of FIG. 1 shows a student (30) downloading and installing the student application (40) and subsequentially assessing the application (40) to establish an account and student profile (280), as further detailed in FIG. 5. Segment 600 of FIG. 1 illustrates how the students (30A, 30B, 30C, 30D) may utilize the student application (40) to undertake a test or assessment (310) and the subsequent computer-implemented analysis of the student's answers, as further detailed in FIG. 6. Finally, segment 700 of FIG. 1 illustrates the review of class and student results (320) by the teacher/administrator (60) based upon completion of the test or assessments by the students and the analysis of the answers/response provided, as detailed in FIG. 7.

As mentioned above, FIG. 2 shows in greater detail the segment 200 of FIG. 1, and in particular FIG. 2 shows the server component (20) which includes infrastructure (10) upon which the platform of the present invention operates. The infrastructure (10) may be local or cloud-based. The central server may operate one or more computer processors and maintain one or more databases to enable the following functionality and/or storage:
Student account register (100) and teacher/administrator account register (110) storing details of students (30) and teachers/administrators (60) who are registered to use the software applications (40) and (70), respectively (e.g. name, address, contact details, details relating to any relevant education institution associated with the student and/or teacher, and any additional data which may be relevant for the purpose of identifying each user). Where possible these details may be verified using identification verification services;
Student profile database (120) storing profiles created by or on behalf of each registered student (30), the profiles containing basic information including some of the information input by the students at the time of creating a user account (including name, age, location and school or education institution), as well as additional details including historical information such as past education institutions, assessments and educational achievements;
Assessment database (130) storing details relating to tests and assessments created by teachers/administrators (60) that will be used to test students (30) to determine their level of knowledge or competence. The assessments may include a series of questions and answers, and cover a range of types, subjects, and may be categorized according to age level. The storage of such assessments may enable teachers/administrators to re-use and modify existing assessments to better conform with their syllabus;
Education institution database (140) containing details relating to schools or other educational institutions as recorded by students (30) and/or each of the teachers/administrators (60) registered to use the applications (40/70);
Assessment answer and results database (160) storing answers provided by students (30) in response to the assessments. As will become apparent, the type of answer that is captured will depend upon the functionality of the device (50) utilized by the student (30), e.g. the answer may include a selected option, a text or numeric answer in writing, and/or a voice capture. For example, a voice capture (162) may occur during a pronunciation activity. A handwritten input (164) may be captured during a writing test in which the student's handwriting or graphical skills are being tested. In another example, typed or selected answers (166) may be input using a keyboard or mouse selection. Database (160) may be in the form of a data structure that appropriately indexes the different types of captured answers against each student (30);
Artificial intelligence analysis engine (170) representing functionality of the server (20) to analyze the answers and results (160) associated with each student (30) to identify students that have achieved success or failure and/or appear to have learning difficulties as compared with a pre-defined threshold or benchmark;
Results/analysis database (180) storing results of analysis carried out by the artificial intelligence engine (170) as well as results from any additional analysis conducted with respect to the performance of any one student (30); and
Alert functionality (190) which automatically generates alerts and/or notifications to teachers/administrators (60) and/or students (30) including, for example when the application (40) identifies that a particular student (30) is having difficulties. An alert may be generated and sent to the software application (70) operating on a device (50) associated with a particular teacher/administrator, or a parent/guardian, for the purpose of enabling the assessor to provide early intervention and assistance to prevent the particular student (30) from falling behind. Such notifications may be a push notification via the software application (40/70), email, text message, or similar.

FIG. 2 also depicts that server (20) is configured to enable communication (210) with the student devices (50) and, in particular, the software application (40) operating on each student device (50). FIGS. 2 and 3 also shows how server (20) is configured to communicate (220) with the teacher/administrator devices (50) and, in particular, with the software application (70) operating on each teacher/administrator device (50). Such communication may occur via the internet or similar network.

FIG. 3 shows in greater detail segment 300 of FIG. 1 and, in particular, the steps associated with the administrators and/or teachers (60) utilizing the software application (70) to create and maintain a range of assessments (e.g. tests, activities, etc) using software application (70) for use by students (40) who operate the corresponding application (40) on their devices (50). The assessments may cover a range of types, subjects and may be directed towards students of different age groups.

It is to be understood that the server (20) may utilize data stored in any one or more of the above-described databases and repositories, or any external data source, regarding the expected educational proficiency for individual students of different ages. This data may be utilized to establish an expected proficiency (i.e. threshold or benchmark) against which to compare an actual education proficiency of each student determined from the assessments. The server (20) may be further operable to adjust assessments automatically in order to suit the educational proficiency of individual students according to individual requirements, disabilities or learning disorders of the students as identified by the system.

FIG. 4 shows in greater detail segment 400 of FIG. 1 and, in particular, the steps associated with a teacher (60) accessing the application (70) to manage and maintain a class profile, as shown in interface (240). The class profile may contain details relating to all students in the particular class managed by the teacher/administrator (70), or year level, and who will undergo the same assessment. The class profile interface (240) may allow the teacher/administrator (70) to edit details, including to add or remove students, and will enable teachers (60) to filter through a list of students in order to locate particular students, view student profiles, and access completed assessments, reports and learning analyses associated with students (30).

The application (70) may also enable, for example via interface (250) shown in FIG. 4, the teacher (60) to schedule an assessment of some or all of the students in the class. In this regard, the teachers (60) may select an assessment based upon the subject and/or year level, and the test or assessment may be either scheduled for completion in class time or the students (30) may be notified to complete the test or assessment for homework.

FIG. 5 shows in greater detail segment 500 of FIG. 1 and, in particular, the steps associated with a student (30) installing (260) the software application (40) on their device (50), and subsequently accessing a user login and registration interface (270) associated with the application (40). Such access may be granted after the student (30) has installed the application (40) which may be achieved by downloading the application (40) from an application store. Each student (30) may create an account using the application (40) and the account information may be stored in the student account register (100).

As described above, the user student account register (100) may capture information sufficient to enable each student to be correctly identified. Alternatively, a student's account may be set up by the educational institution responsible for the student for and on behalf of the student (30).

Once the application (40) has been accessed by student (30), the student (30) may be presented with an interface (280) that allows the student (30) to create and maintain a student profile that will subsequently enable students (30) (as well as teachers/administrators (60)) to view information about the student (30), and further allow students (30) to manage their student profile. The profile may contain basic information about the student including name, age, location, school, etc. and over time, the student's profile may include additional information such as a history of past schools/institutions, assessment and educational achievements. The student profile may also include information relating to any learning difficulties or disorders associated with the student (30), as determined by the system. Such difficulties or disorders may include, but are not limited to, dyslexia, dysgraphia, dyscalculia, auditory process disorders, language processing disorders, non verbal learning difficulties and visual perceptional, visual motor deficits.

FIG. 6 shows in greater detail segment 600 of FIG. 1 and, in particular, the steps associated with a student (30) using application (40) to undertake a test or assessment. Shown in FIG. 5 is a classroom (290) including a teacher (60) and four students (30A, 30B, 30C and 30D). Each student is operating a device (50) upon which the software application (40) is operating, enabling each student (30) to provide input via an assessment interface (310) for example. The assessment interface (310) may be presented in one of several different ways depending upon the particular test or assessment, e.g. a range online questions and/or activities. The software application (40) will capture the student's answers to questions and activities.

The type of answer captured will depend upon the particular device capabilities, and the assessment question/activity. For example, answers may be input by way of selecting an option that is presented on the interface (310), writing text or numeric answers, hand-writing or voice capture. For example, FIG. 6 shows that software application (40) operating on device (50) is capable of voice capture (312) (e.g. using a microphone or similar component on device 50), handwriting capture (314) (e.g. capturing handwriting by the use of a stylus, electronic pen or similar component on the device (50), and the capture (316) of typed or selected answers (e.g. answers input by the student (30), mouse, touchpad or similar component not on the device (50).

Accordingly, it will be appreciated that depending upon the classroom arrangements, students may either be notified by the application (40) or requested by their teacher (60) to take an assessment that the teacher (60) has scheduled on the application. The student (30) may locate the assessment from a listing of assessments to be completed, for example, and the application (40) may then assess the student (30) through a range of online questions and/or activities. The application (40) will capture the student's answers/response to questions and activities, and the type of answers/response captured will depend upon the device capabilities, as described above.

The analysis of a student's answers/response to questions and/or activities may be carried out by the artificial intelligence analysis engine (170), which may implement one or more algorithms that in addition to assessing an education proficiency of the student (30) is also operable to determine the presence of one or more indicators regarding learning difficulties and/or disorders associated with the student (30). For example, the one or more indicators may relate to learning difficulties and/or disorders with respect to reading, spelling or mathematics. In the case where the data received from the student (30) is in the form of a voice recording, the analysis may involve the implementation of voice recognition algorithms such that word and sound pronunciation may be assessed. Additional algorithms may be implemented so that an appropriate analysis of the student's answers/response may be undertaken, including for example, a reading, spelling or mathematical analysis algorithm to determine the educational proficiency of the student (30).

The algorithms may operate on the basis of benchmarks or thresholds, that is, whereby particular answers or responses from each student (30) are compared against the particular benchmark or threshold associated with a particular question or activity. Where the student's answers/response satisfies the benchmark or threshold, a particular educational proficiency level (as well as a particular learning difficulty and/or disability) may be determined and the particular student may be categorized accordingly. For example, based on the analysis results, it may be determined that student (30A) has an acceptable educational proficiency with no learning difficulties or disabilities, whilst student (30B) is found not to satisfy the particular benchmark or threshold associated with a particular question or activity and is therefore determined as having an educational proficiency that requires additional monitoring or assistance. It may further be further determined that student (30C) has a particular learning difficulty and/or disability based on the analysis of the answers/response provided by student (30C) during a single assessment, or based upon the answers/response provided over multiple assessments.

It will be appreciated that by collecting data regarding online learning assessments and analyzing same in this manner, a record of a student's progress along their learning journey may be recorded, providing a useful repository of data for identifying learning disabilities that are otherwise difficult to identify and assess in situations that require consistent and regular monitoring of a student's progress over an extended period of time, e.g. over a number of years. The sharing of such data may be particularly useful if a student transfers from one school or institution to another or transitions from primary school to high school to tertiary school.

FIG. 7 shows in greater detail segment 700 of FIG. 1 and, in particular the steps associated with the review of class and student results by the teacher (60), including the ability to access student reports and for the teacher (60) and student (30) to receive notifications. Shown in FIG. 7 is an example interface (320) accessible by the teacher (60) using software application (70) to access their student's results and learning analysis as they complete their assessments (e.g. the analysis results output by artificial intelligence engine (170)). Students who have performed poorly or have been identified by the application (70) as having learning difficulties and/or disorders may be highlighted (322) in interface (320) to enable the teacher (60) to more easily identify those instances.

The teacher (60) may select a particular student from the listing in order to be provided with additional information relating to the student's answers and results. In this regard, interface (330) is an example interface provided to teacher's (60) to enable teachers (60) to view a detailed report on the selected students progress based upon assessments (130) and the artificial intelligence analysis (170). The report may also show assessment results (160), as well as trends and any identified learning difficulties (190). As required, such reports may be printed and shared with parents and students to indicate progress and determine when interventions may be required.

Both software applications (40/70) may generate alerts and/or notifications to the teachers (60) as well as to the students (30). For example, the notifications may identify notable events with respect to the assessment of a student (30), such as a higher than average result with respect to a particular test or assessment, or on the other hand a lower than average result.

An additional benefit of storing the various inputs by students (30) in response to questions and activities, and the results of analysis conducted by the artificial intelligence engine (170) and any additional processing techniques performed by server (20) with respect to the assessment of students and their learning difficulties and/or disorders, is that the server (20) may subsequently be used to conduct various additional analyses based on historical data, such as trend analyses and the like. A trend analysis with respect to student assessment may be used to determine individual student progress and identify trends that will result in difficulty for a student to achieve an expected educational proficiency in the future. This allows teachers/administrators as well as parents and/or guardians to identify potential learning disabilities/disorders without the need for significant events to be identified, since through the use of the application (70) a student's educational proficiency may be followed/monitored over a sufficient period of time in an objective and consistent manner. Therefore, indicators of any disabilities and/or learning disorders associated with students (30) that may otherwise be imperceptible to an assessor, may now become more apparent through the identification of trends in the students progress. This may enable early intervention and increased prospects for better learning outcomes and student academic progress.

As used herein, the term “server”, “system”, “computer”, “computing system” or the like may include any processor-based or microprocessor-based system including systems using microcontrollers, reduced instruction set computers (RISC), application specific integrated circuits (ASICs), logic circuits, and any other circuit or processor including hardware, software, or a combination thereof capable of executing the functions described herein. Such are exemplary only, and are thus not intended to limit in any way the definition and/or meaning of such terms.

The one or more processors as described herein are configured to execute a set of instructions that are stored in one or more data storage units or elements (such as one or more memories), in order to process data. For example, the one or more processors may include or be coupled to one or more memories. The data storage units may also store data or other information as desired or needed. The data storage units may be in the form of an information source or a physical memory element within a processing machine.

The set of instructions may include various commands that instruct the one or more processors to perform specific operations such as the methods and processes of the various embodiments of the subject matter described herein. The set of instructions may be in the form of a software program. The software may be in various forms such as system software or application software. Further, the software may be in the form of a collection of separate programs, a program subset within a larger program or a portion of a program. The software may also include modular programming in the form of object-oriented programming. The processing of input data by the processing machine may be in response to user commands, or in response to results of previous processing, or in response to a request made by another processing machine.

The diagrams of embodiments herein illustrate one or more control or processing units. It is to be understood that the processing or control units may represent circuits, circuitry, or portions thereof that may be implemented as hardware with associated instructions (e.g., software stored on a tangible and non-transitory computer readable storage medium, such as a computer hard drive, ROM, RAM, or the like) that perform the operations described herein. The hardware may include state machine circuitry hardwired to perform the functions described herein. Optionally, the hardware may include electronic circuits that include and/or are connected to one or more logic-based devices, such as microprocessors, processors, controllers, or the like.

Optionally, the one or more processors may represent processing circuitry such as one or more of a field programmable gate array (FPGA), application specific integrated circuit (ASIC), microprocessor(s), and/or the like. The circuits in various embodiments may be configured to execute one or more algorithms to perform functions described herein. The one or more algorithms may include aspects of embodiments disclosed herein, whether or not expressly identified in the figures or a described method.

It will be appreciated by persons skilled in the relevant field of technology that numerous variations and/or modifications may be made to the invention as detailed in the embodiments without departing from the spirit or scope of the invention as broadly described. The present embodiments are, therefore, to be considered in all aspects as illustrative and not restrictive.

Throughout this specification and claims which follow, unless the context requires otherwise, the word “comprise”, and variations such as “comprises” and “comprising”, will be understood to imply the inclusion of a stated feature or step, or group of features or steps, but not the exclusion of any other feature or step or group of features or steps.

1. A computer-implemented system for facilitating the collection and analysis of academic assessment data, the system including:
one or more processors in operable communication with one or more devices, each device operable by a student and configured to connect with a data communications network such that any information requested of, or received by, the student, is retrieved from, and/or stored in, a data repository connected to the data communications network;
the one or more processors operable to:
request information from the student in accordance with an assessment;
receive data entered by the student using their device in response to the request for information;
retrieve data stored in the data repository associated with the student;
perform analysis upon the received data from the student and the retrieved data associated with the student to determine the educational proficiency of the student according to the assessment and further assessing the presence of one or more indicators regarding one or more learning disorders associated with the student; and
generate a report regarding the educational proficiency and/or presence of the one or more indicators for subsequent storage and/or transmission.

2. A computer-implemented system according to claim 1, wherein the results of the performed analysis are collected and stored in the data repository for sharing with, and/or accessible by, other assessors and/or individuals.

3. A computer-implemented system according to either claim 1 or claim 2, wherein the analysis includes the implementation of one or more algorithms operable to determine the presence of the one or more indicators regarding the one or more learning difficulties or disorders associated with the student.

4. A computer-implemented system according to any one of the preceding claims, wherein the one or more indicators relate to one or more learning difficulties or disorders with respect to reading, spelling and mathematics.

5. A computer-implemented system according to any one of the preceding claims, wherein the data collected by the system includes recorded voice data, and the analysis includes voice recognition algorithms for assessment of word and sound pronunciation.

6. A computer-implemented system according to any one of the preceding claims, wherein the data stored in the data repository is available for review by the student and/or assessors.

7. A computer-implemented system according to any one of the preceding claims, wherein the system performs analysis to determine the presence of indicators regarding learning difficulties or disorders including any one or more of dyslexia, disgraphia, dyscalcula, auditory processing disorders, language processing disorders, nonverbal learning disabilities and visual perceptual/visual motor deficit.

8. A computer-implemented system according to any one of the preceding claims, wherein the data repository stores further information regarding the expected education proficiency for individual students of different ages and the one or more processors apply the educational proficiency expected for individual students and compares same with the actual education proficiency of each student determined from the assessments.

9. A computer-implemented system according to any one of the preceding claims, wherein the one or more processors are further operable to adjust assessments to suit the educational proficiency of individual students according to individual requirements, disabilities or learning disorders of the students as identified by the system.

10. A computer-implemented system according to any one of the preceding claims, wherein the one or more processors are operable to provide assessors and/or parents/guardians of a student with one or more notifications identifying notable events with respect to the assessment of the student.

11. A computer-implemented system according to any one of the preceding claims, wherein the one or more processors are operable to conduct one or more trend analyses with respect to student assessment in order to determine individual student progress and identify trends that will likely result in difficulty for a student to achieve an expected educational proficiency.

12. A computer-implemented system according to any one of the preceding claims, wherein the system is further operable to enable students and/or assessors to access the system from any geographical location and at any time.

13. A computer-implemented method for facilitating the collection and analysis of academic assessment data, the method including:
requesting, by one or more processors associated with a computer-implemented system, information from a student in accordance with an assessment;
receiving, by the one or more processors, data entered by the student in response to the request for information using a device operable to connect with a data communications network and storing the data in a data repository connected to the data communications network,
retrieving, by the one or more processors, the data associated with the student stored in the data repository;
performing, by the one or more processors, an analysis upon the data associated with the student to determine the educational proficiency of the student according to the assessment and further assessing the presence of one or more indicators regarding one or more learning disorders associated with the student; and
generating, by the one or more processors, a report regarding the educational proficiency and/or presence of the one or more indicators for subsequent storage and/or transmission.

14. A computer-implemented method according to claim 13, wherein the results of the performed analysis are collected and stored in the data repository for sharing with, and/or accessible by, other assessors and/or individuals.

15. A computer-implemented method according to either claim 13 or claim 14, wherein the analysis includes the implementation of one or more algorithms operable to determine the presence of the one or more indicators regarding the one or more learning difficulties or disorders associated with the student.

16. A computer-implemented method according to any one of claims 13 to 15, wherein the one or more indicators relate to one or more learning difficulties or disorders with respect to reading, spelling and mathematics.

17. A computer-implemented method according to any one of claims 13 to 16, wherein the data collected, by the one or more processors, includes recorded voice data, and the analysis includes voice recognition algorithms for assessment of word and sound pronunciation.

18. A computer-implemented method according to any one of claims 13 to 17, wherein the data stored in the data repository is available for review by the student and/or assessors.

19. A computer-implemented method according to any one of claims 13 to 18, wherein the method further includes performing, by the one or more processors, an analysis to determine the presence of indicators regarding learning difficulties or disorders including any one or more of dyslexia, disgraphia, dyscalcula, auditory processing disorders, language processing disorders, nonverbal learning disabilities and visual perceptual/visual motor deficit.

20. A computer-implemented method according to any one of claims 13 to 19, wherein the method further includes storing, by the one or more processors, information regarding the expected education proficiency for individual students of different ages and the one or more processors apply the educational proficiency expected for individual students and compares same with the actual education proficiency of each student determined from the assessments.

21. A computer-implemented method according to any one of claims 13 to 20, wherein the method further includes adjusting, by the one or more processors, assessments to suit the educational proficiency of individual students according to individual requirements, disabilities or learning disorders of the students as identified by the system.

22. A computer-implemented method according to any one of claims 13 to 21, wherein the method further includes providing, by the one or more processors, assessors and/or parents/guardians of a student with one or more notifications identifying notable events with respect to the assessment of the student.

23. A computer-implemented method according to any one of claims 13 to 22, wherein the method further includes conducting, by the one or more processors, one or more trend analyses with respect to student assessment in order to determine individual student progress and identify trends that will likely result in difficulty for a student to achieve an expected educational proficiency.

24. A computer-implemented method according to any one of claims 13 to 23, wherein the method further includes enabling, the by one or more processors, students and/or assessors to access the stored data and information in the data repository from any geographical location and at any time.

25. A computer-readable medium that when executed on a computer, causes one or more processors of the computer to perform the steps of:
request information from a student in accordance with an assessment;
receive data entered by the student in response to the request for information using a device operable to connect with a data communications network and storing the data in a data repository connected to the data communications network,
retrieve the data associated with the student stored in the data repository;
perform an analysis upon the data associated with the student to determine the educational proficiency of the student according to the assessment and further assess the presence of one or more indicators regarding one or more learning disorders associated with the student; and
generate a report regarding the educational proficiency and/or presence of the one or more indicators for subsequent storage and/or transmission.
